(19) United States
(12) Patent Application Publication
(10) Pub. No.: US 2018/0042256 A1
(43) Pub. Date: Feb. 15, 2018

(54) NOVEL THERMOTOLERANT LACTOBACILLUS

(71) Applicant: CHR. HANSEN A/S, Hoersholm (DK)

(72) Inventors: Gaelle Lettier BUCHHORN, Virum, (DK); Dina VANELL, VCopenhagen Ø (DK); Kim lb SOERENSEN, Farum (DK); Gunnar OEREGAARD, Vaerloese (DK); Annette KIBENICH, Hvidovre (DK); Per STTROEMAN, Naerum (DK)

(73) Assignee: Chr. Hansen A/S, Hoersholm (DK)

(21) Appl. No.: 15/552,398

(22) PCT Filed: Feb. 22, 2016
(86) PCT No.: PCT/EP2016/053664

§ 371 Date: Aug. 21, 2017

(30) Foreign Application Priority Data
Feb. 23, 2015 (EP) 15156057.0

(51) Int. Cl. A23C 19/032; C12N 1/20; C12R 1/225

(52) U.S. Cl. CPC A23C 19/0323; C12R 1/225; C12N 1/20; A23Y 2220/29

(57) ABSTRACT

The present invention relates to a high temperature resistant Lactobacillus delbrueckii subsp. lactis DSM 32009 strain that can be used in the production of cheese where the curd is cooked at high temperature (>50° C.).

### FIELD OF INVENTION

The present invention relates to a thermotolerant Lactobacillus delbrueckii subsp. lactis DSM 32009 strain that can be used in the production of cheese where curd cooking at high temperature (>50° C.) is a part of the cheese manufacturing process, such as e.g. Grana and Emmental cheese.

### BACKGROUND OF INVENTION

During cheese manufacture the curd-whey mixture is often heated in order to promote syneresis. The step is referred to as scalding or cooking. The temperature used is different for each cheese variety. For cheeses such as Emmental and Parmesan this temperature is high, usually in the range of 52 to 55° C. Such high temperatures can have a negative consequence on syneresis and cheese quality as they can kill or strongly inhibit the growth of the starter culture, thus resulting in poor acidification of the curd. For these types of cheeses, it is important that the starter culture is composed of thermophilic strains that can survive the high temperature cooking step. Strains that can grow and acidify at high temperature would also be a clear advantage.

### SUMMARY OF INVENTION

To develop bacterial strains that are able to acidify milk even at temperatures above 50° C. and therefore can be used for cheese making processes with a high temperature cooking step (>50° C.) a screening process was designed and executed. The outcome of the screening was surprising as only one strain was able to perform satisfactory in the temperature profile used for the production of whey starter for manufacture of Grana Padano cheese. This strain was identified to be a Lactobacillus delbrueckii subsp. lactis strain.

It was further observed that only one isolate of the tested strains, Lactobacillus delbrueckii subsp. lactis can acidify milk at a temperature 50° C. and consequently perform satisfactory in the temperature profile used for the production of whey starter for manufacture of Grana Padano cheese. The strain is assigned the deposit number: DSM 32009.

In accordance with the surprising finding, an aspect of the present invention relates to the use of a Lactobacillus delbrueckii subsp. lactis DSM 32009 strain, that can acidify milk at high temperature, in a cheese making process that involves a high temperature cooking step (>50° C.).

### DETAILED DESCRIPTION OF THE INVENTION

The present invention relates to a process for producing cheese, which comprises adding to milk a starter culture comprising an acidifying strain belonging to the genus Lactobacillus and heating the mixture to a temperature (or maintaining the temperature) in the range of 45° C. to 65° C., such as in the range 50° C. to 60° C. or in the range 53-54° C., such as e.g. 54° C.

In a related aspect, the present invention relates to a process for producing cheese, which comprises adding to milk a starter culture comprising an acidifying strain belonging to the genus Lactobacillus and heating the mixture to a temperature (or maintaining the temperature) in the range of 45° C. to 65° C., such as in the range 50° C. to 60° C. or in the range 53-54° C., such as e.g. 54° C. and adding to mixture one or more bacterial strains or cultures, such as e.g. adjunct cultures and optionally one or more coagulants.

In an embodiment of present invention, the acidifying Lactobacillus is able to acidify the milk by at least 0.4 pH units in 2.5 hours at a temperature around 50° C. when inoculated with a 3% whey starter culture.

In a related aspect, the acidifying strain of Lactobacillus is a Lactobacillus delbrueckii, such as e.g. Lactobacillus delbrueckii subsp. lactis, optionally the strain deposited as DSM 32009, or a mutant or variant thereof.

In a related aspect, the starter culture further comprise one or more bacterial strains selected from the list consisting of: Lactococcus lactis, Leuconostoc mesenteroides, Pediococcus pentosaceus, Lactobacillus casei, Lactobacillus paracasei, Streptococcus thermophilus, Enterococcus faecium, Lactobacillus delbrueckii subsp. bulgaricus, Lactobacillus delbrueckii subsp. lactis, Lactobacillus helveticus spp, Lactobacillus acidophilus or Propionibacterium.

In yet a related aspect, the one or more coagulants is selected from the list comprising: bovine derived coagulants or variants thereof, microbial derived coagulants or variants thereof, camel derived coagulants or variants thereof, rennets and chymosins.

In yet a related aspect, the starter culture is a whey starter culture and in related embodiments the whey starter culture may be subject to the physical conditions comprising: 1) a temperature drop from around 48° C. to around 39° C. in 9 hours, 2) a constant temperature of around 39° C. for around 8 hours, 3) cooling and storing at around 16° C. until it is used for milk acidification.

In yet a related aspect, the starter culture and or one or more bacterial strains is added as a direct vat set (DVS) culture, such as e.g. a frozen direct vat set (F-DVS) culture or a freeze-dried direct vat set (FD-DVS) culture. The DVS culture may be added to a whey starter and/or directly to the milk. If added to the whey, the acidifying strain belonging to the genus Lactobacillus may be added in an amount of at least 5×10¹⁰ CFU/500 liter whey.

The process of present invention may further comprise adding one or more bacterial strains or cultures, such as e.g. adjunct cultures such as e.g. Propionibacterium to the milk.

The process of present invention may further comprise the necessary actions needed to produce a cheese. Hence the present invention also relate to a cheese (including a low-fat cheese) obtainable by the process of any of the aspect disclosed herein. More specifically the may be a Grana type cheese such as e.g. a Grana Padano, Parmigiano Reggiano or alternatively of the Emmental type.

As disclosed herein, the invention also relate to an acidifying Lactobacillus delbrueckii subsp. lactis strain, such as e.g. a Lactobacillus delbrueckii subsp. lactis strain any mutant or variant thereof and optionally the Lactobacillus delbrueckii subsp. lactis strain deposited as DSM 32009 or any mutant or variant thereof. If commercially relevant, the acidifying strain of Lactobacillus may be modified by the act of man.

The Lactobacillus of present invention may be forming part of a mixture with desired properties. Hence the present invention also relates to a composition comprising the acidifying Lactobacillus delbrueckii as described in present disclosure. Such composition may comprise one or more excipients such as e.g. polyhydroxy compounds such as e.g. sugars, polyalcohols and glycols and their derivatives, trehalose and dimethyl sulfoxide (DMSO).

The composition of present invention may be a direct vat set (DVS) starter culture composition, such as e.g. a frozen direct vat set (F-DVS) starter culture composition or a freeze-dried direct vat set (FD-DVS) starter culture composition. The DVS starter culture may be added to a whey starter. In a related aspect, the DVS starter culture composition comprises at least 1×10¹⁰ CFU/g.

In a related aspect, the starter culture is added to the milk in an amount sufficient to facilitate acidification of the milk by at least 0.4 pH units in 2.5 hours at a temperature around 50° C.

As illustrated by the vast aspects described herein, the invention also relate to the use of an acidifying Lactobacillus delbrueckii subsp. lactis strain (e.g. DSM32009) in a process for making cheese, such as e.g. Grana or Emmental type cheese as well as a composition as described in the aspects herein, in a process for making cheese, such as e.g. Grana or Emmental type cheese.

Hence, in summary, the present invention relates to the following related aspects

Aspect 1. A process for producing cheese, which comprises adding to milk:
a starter culture comprising an acidifying strain belonging to the genus Lactobacillus and
   heating the mixture to a temperature (or maintaining the temperature) in the range of 45 to 65° C., such as in the range 50 to 60° C. or in the range 53-54° C., such as e.g. 54° C.

Aspect 2. A process according to aspect 1, wherein the acidifying Lactobacillus is able to acidify the milk by at least 0.4 pH units in 2.5 hours at a temperature around 50° C. when inoculated with a 3% whey starter culture.

Aspect 3. A process of any preceding aspect, wherein the acidifying strain of Lactobacillus is a Lactobacillus delbrueckii or a mutant or variant thereof.

Aspect 4. A process of any preceding aspect, wherein the acidifying strain of Lactobacillus is a Lactobacillus delbrueckii subsp. lactis or a mutant or variant thereof.

Aspect 5. A process of any preceding aspect, wherein the acidifying strain of Lactobacillus is a Lactobacillus deposited as DSM 32009 or a mutant or variant thereof.

Aspect 6. A process of any preceding aspect, wherein the acidifying strain of Lactobacillus is modified by the act of man.

Aspect 7. A process of any preceding aspect, further comprising adding to milk
one or more bacterial strains or cultures, such as e.g. adjunct cultures.

Aspect 8. A process according any of aspects 1 to 7, wherein the starter culture comprise one or more bacterial strains selected from the list consisting of: Lactococcus lactis, Leuconostoc mesenteroides, Pediococcus pentosaceus, Lactobacillus casei, Lactobacillus paracasei, Streptococcus thermophilus, Enterococcus faecium, Lactobacillus delbrueckii subsp. bulgaricus, Lactobacillus delbrueckii subsp. Lactis, Lactobacillus helveticus spp, Lactobacillus delbrueckii, Lactobacillus acidophilus and Propionic bacterium.

Aspect 9. A process according to aspect 8 wherein the starter culture comprise Lactobacillus helveticus spp, Lactobacillus delbrueckii, Streptococcus thermophilus and Lactobacillus casei.

Aspect 10. A process of any preceding aspect, further comprising adding to milk
one or more coagulants.

Aspect 11. A process according to aspect 10 wherein the one or more coagulants is selected from the list comprising: bovine derived coagulants or variants thereof, microbial derived coagulants or variants thereof, camel derived coagulants or variants thereof, rennets and chymosins.

Aspect 12. A process of any of the preceding aspects, wherein the starter culture is a whey starter culture.

Aspect 13. A process according to aspect 12 wherein the whey starter culture is subject to the physical conditions comprising: 1) a temperature drop from around 48° C. to around 39° C. in 9 hours, 2) a constant temperature of around 39° C. for around 8 hours, 3) cooling and storing at around 16° C. until it is used for milk acidification.

Aspect 14. A process according to any of the preceding aspects wherein the starter culture and or one or more bacterial strains is added as a direct vat set (DVS) culture, such as e.g. a frozen direct vat set (F-DVS) culture or a freeze-dried direct vat set (FD-DVS) culture.

Aspect 15. A process according to aspect 14, wherein the DVS culture is added to a whey starter.

Aspect 16. A process according to any of the preceding aspects, wherein the acidifying strain belonging to the genus Lactobacillus is added in an amount of at least 5×10¹⁰ CFU/500 liter whey.

Aspect 17. A process according to aspect 14, wherein the DVS culture is added directly to the milk.

Aspect 18. A process according to any of aspects 1 to 17, wherein the starter culture is added to the milk in an amount sufficient to facilitate acidification of the milk by at least 0.4 pH units in 2.5 hours at a temperature around 50° C.

Aspect 19. A process according to aspect 7, wherein the one or more bacteria belongs to the genus Propionibacterium.

Aspect 20. A cheese (including a low-fat cheese) obtainable by the process of any preceding aspects.

Aspect 21. A cheese according to aspect 20, wherein the cheese is a Grana type cheese such as e.g. a Grana Padano, Parmigiano Reggiano or of the Emmental type.

Aspect 22. An acidifying Lactobacillus delbrueckii subsp. lactis strain.

Aspect 23. An acidifying Lactobacillus delbrueckii according to aspect 22, wherein the Lactobacillus delbrueckii strain is an acidifying Lactobacillus delbrueckii subsp. lactis strain desposited as DSM 32009 or a mutant or variant of the acidifying Lactobacillus delbrueckii subsp. lactis strain desposited as DSM 32009.

Aspect 24. An acidifying Lactobacillus delbrueckii strain according to aspect 22 or 23 wherein the acidifying Lactobacillus delbrueckii strain is able to acidify the milk by at least 0.4 pH units in 2.5 hours at a temperature around 50° C. when inoculated with a 3% starter culture.

Aspect 25. An acidifying Lactobacillus delbrueckii strain according to aspect 23 to 24, wherein the acidifying strain of Lactobacillus is modified by the act of man.

Aspect 26. A composition comprising the acidifying Lactobacillus delbrueckii strain of any of aspects 20 to 25.

Aspect 27. A composition according to aspect 26 further comprising one or more excipients.

Aspect 28. A composition according to aspect 27, wherein the one or more excipients is selected from a list comprising: polyhydroxy compounds such as e.g. sugars, polyalcohols and glycols and their derivatives, trehalose and dimethyl sulfoxide (DMSO).

Aspect 29. A composition according to aspect 27 or 28, wherein the composition is a direct vat set (DVS) starter culture composition, such as e.g. a frozen direct vat set (F-DVS) starter culture composition or a freeze-dried direct vat set (FD-DVS) starter culture composition.

Aspect 30. A composition according to any of aspects 26 to 29, wherein the DVS starter culture composition comprise at least 1×10̂10 CFU/g.

Aspect 31. Use of an acidifying Lactobacillus delbrueckii subsp. lactis strain of any of aspects 22 to 25 in a process for making cheese, such as e.g. Grana or Emmental type cheese.

Aspect 32. Use of a composition according to any of aspects 26 to 30 in a process for making cheese, such as e.g. Grana or Emmental type cheese.

### Definitions

By the term “milk” is understood a composition comprising lacteal secretion obtained from any mammal, such as an animal of a species belonging to the subfamily Bovinae (which includes the domestic cow (Bos taurus) and buffalo); an animal of a species belonging to the subfamily Caprinae (which includes goat and sheep); or an animal of the species Camelidae (which includes camels). Optionally the milk is acidified, e.g. by addition of an acid (such as citric, acetic or lactic acid) or by addition of an acid producing microorganism. The milk may be raw or processed, e.g. by filtering, sterilizing, pasteurizing, homogenizing, fractionating (e.g. reducing the fat content of the milk), etc., or it may be reconstituted dried milk. An important example of “milk” according to the present invention is pasteurized cow's milk. It is understood that the milk may be acidified, mixed or processed before, during and/or after the adding of bacterial cultures. The term “milk” also comprises milk with protein, calcium or other additives added.

The term “cheese” refers to a product prepared by contacting optionally acidified milk (e.g. by means of a lactic acid bacterial culture) with a coagulant, and draining the resultant curd. Cheeses and their preparation are described in e.g. Cheese and Fermented Milk Foods, by Frank V. Kosikowski.

The term “high temperature cooking step” should be understood as a cooking step with a temperature >50° C. This is typical for e.g. cheese of the Grana and Emmental type.

As used herein the term ‘modified by the act of man’ should be understood as a modification of the Lactobacillus strain aiming to increase the commercial applicability of said Lactobacillus strain. Such methods comprise increasing the thermo-tolerance by growing the strain at elevated temperatures such as e.g. >50° C., subjecting the strain to heat-stress to cure antibiotic resistance, such as e.g. kanamycin resistance if relevant. Other modifications made by man could be optimization for accelerated germination or acidification.

As used herein the term “lactic acid bacterium” designates a gram-positive, microaerophilic or anaerobic bacterium, which ferments sugars with the production of acids including lactic acid as the predominantly produced acid. The industrially most useful lactic acid bacteria are found among Lactococcus spp., Streptococcus spp., Lactobacillus spp., Leuconostoc spp., Pediococcus spp. Additionally, lactic acid producing bacteria belonging to the group of the strict anaerobic bacteria, bifidobacteria, i.e. Bifidobacterium spp., which are frequently used as food cultures alone or in combination with other lactic acid bacteria, are generally included in the group of lactic acid bacteria.

The term “starter culture” relates to any bacterial culture that is suitable for use in milk acidification, especially lactic acid bacteria such as Bifidobacteria, Lactobacilli, Lactococci, Leuconostocs, Micrococci, Streptococci and Pediococci. It will be appreciated that the term starter culture may encompass a culture containing a single strain of bacterium, or more than one bacterial strain. The term may also include genetically modified organisms (GMO's). In any event, the term is well known in the art and the invention extends equally to all known starter cultures. The term includes bacterial cultures containing a strain of a genus selected from the group consisting of Lactococcus, Lactobacillus, Micrococcus, Leuconostoc, Pediococcus, Streptococcus, Enterococcus, etc. such as a strain of a species selected from the group consisting of: Lactococcus lactis (incl. Lactococcus subsp. lactis subsp. lactis, Lactococcus lactis subsp. cremoris, and Lactococcus lactis subsp. lactis biovar. diacetylactis), Leuconostoc mesenteroides (incl subsp. cremoris), Pediococcus pentosaceus, Lactobacillus casei (incl. subsp. casei) and Lactobacillus paracasei (incl. subsp. paracasei), Streptococcus thermophilus, Enterococcus faecium, Lactobacillus helveticus, Lactobacillus delbrueckii subsp. bulgaricus, Lactobacillus delbrueckii subsp. subsp. lactis and Lactobacillus acidophilus. Other useful bacterial species are Bifidobacterium species including B. bifidum, B. lactis and B. longum, Streptococcus faecium, Leuconostoc lactis.

As previous mentioned, lactic acid bacteria are essential in the making of nearly all fermented milk products e.g. cheese, and they are normally supplied to the dairy industry either as frozen or freeze-dried cultures for bulk starter propagation or as so-called “Direct Vat Set” (DVS) cultures, intended for direct inoculation into a fermentation vessel or vat for the production of a dairy product. Such cultures are in general referred to as “starter cultures” or “starters”.

Commonly used starter culture strains of lactic acid bacteria are generally divided into mesophilic organisms having optimum growth temperatures at about 30 to 35° C. and thermophilic organisms having optimum growth temperatures in the range of about 40 to about 45° C. Typical organisms belonging to the mesophilic group include Lactococcus lactis subsp. lactis, Lactococcus lactis subsp. cremoris, Leuconostoc mesenteroides subsp. cremoris, Pediococcus pentosaceus, Lactococcus lactis subsp. lactis biovar. diacetylactis, Lactobacillus casei subsp. casei and Lactobacillus paracasei subsp. paracasei. Thermophilic or thermotolerant lactic acid bacterial species include as examples Streptococcus thermophilus, Enterococcus faecium, Lactobacillus delbrueckii subsp. lactis, Lactobacillus helveticus, Lactobacillus delbrueckii subsp. bulgaricus, Lactobacillus delbrueckii subsp. lactis and Lactobacillus acidophilus.

Additionally, species of Propionibacterium are used as dairy starter cultures, in particular in the manufacture of cheese. As reflected by the teachings disclosed in e.g. Rossetti et al. 2008, several studies have been made on the microbial composition of starter cultures in cheese making. Hence the person skilled in the art of making cheese (and in particular Grana) will know the composition of the starter-culture to which the strain according to present invention may be added.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### DRAWINGS

FIG. 1 depicts the temperature profile used for the generation of the whey starter culture containing the Lactobacillus delbrueckii subsp. lactis DSM 32009, cf. the example 1.

FIG. 2 depicts the evolution of the pH in the whey starter solution during the incubation step as described in FIG. 1.

FIG. 3 depicts the temperature profile used for the milk acidification using a whey starter containing the L. delbrueckii subsp. lactis.

FIG. 4 depicts the evolution of the pH in milk inoculated with a whey starter solution with and without the L. delbrueckii subsp. lactis.

### EXPERIMENTAL

### Example 1

### Acidification of Milk by a Whey Starter Containing the Lactobacillus delbrueckii subsp. lactis DSM 32009 Strain when a Temperature Gradient with a High Temperature Cooking Step is Applied.

a) Generation of whey starter containing the Lactobacillus delbrueckii subsp. lactis DSM 32009 strain.

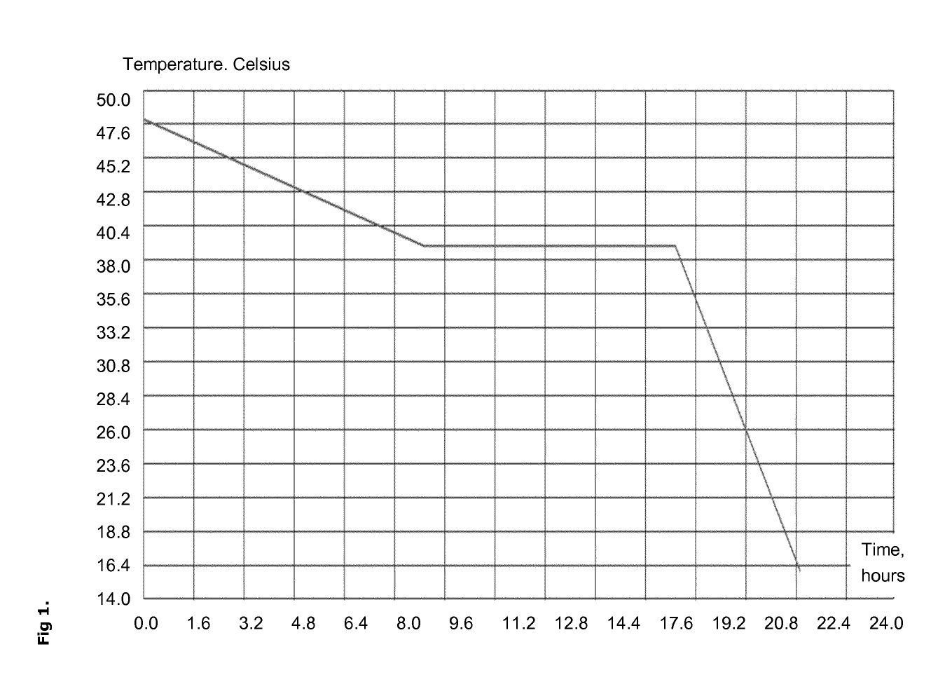

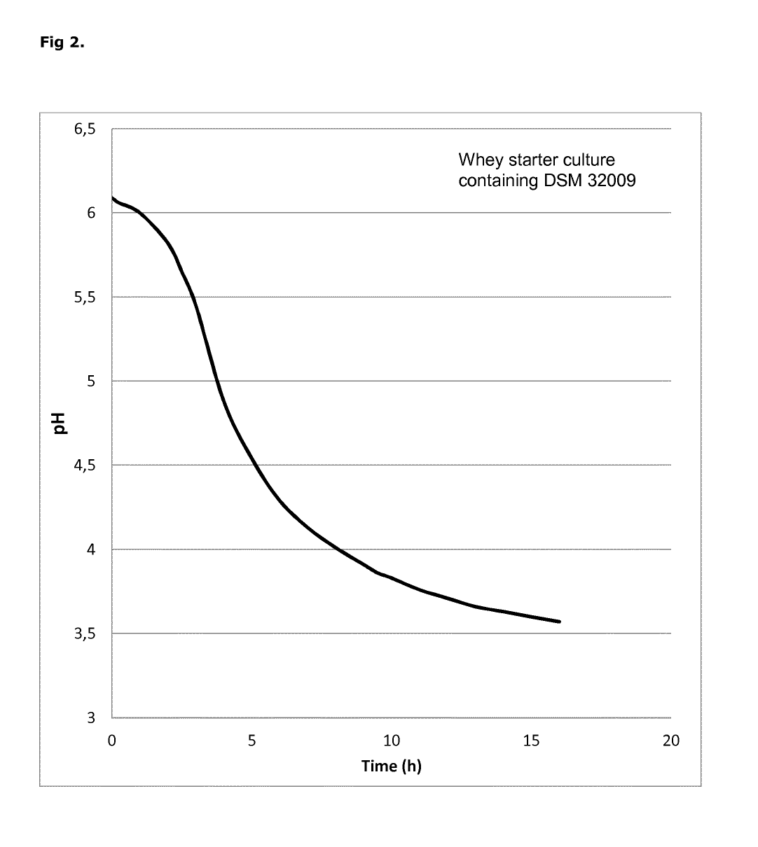

Whey starter media consisted of a 7% sweet whey powder solution in water. After dissolution of the whey powder, the whey media was pasteurized for 20 minutes at 90° C. 200 ml of sweet whey media were inoculated with the whey starter culture comprising a mix of Lactobacillus spp and Streptococcus thermophilus with and without DSM 32009 The starter was incubated as described in FIG. 1. pH was measured continuously with pH probes connected to a datalogger as shown in FIG. 2. Temperature compensation was used to secure accurate pH measurement throughout the temperature gradient. The whey starter culture containing the Lactobacillus delbrueckii subsp. lactis DSM 32009 strain acidified to pH 3.5 in 16 hours.

FIG. 1 shows the temperature profile used for the generation of the whey starter culture. This temperature profile simulates the temperature profile used for the production of whey starter for manufacture of Grana Padano cheese. The temperature profile consists of:
A temperature drop from 48° C. to 39° C. in 9 hours
A constant temperature of 39° C. for 8 hours
The whey starter is then cooled and maintained at 16° C. until it is used for milk acidification.

FIG. 2 shows the evolution of the pH in the whey starter solution during the incubation step as described in FIG. 1.

b) Acidification of milk with a whey starter containing the Lactobacillus delbrueckii subsp. lactis DSM 32009 strain

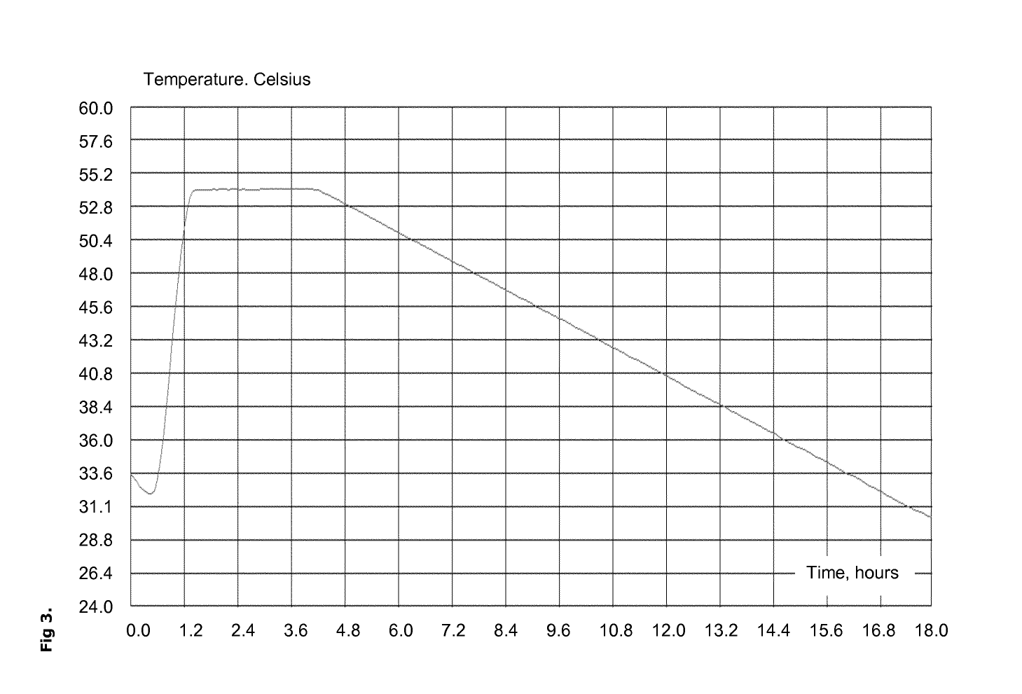

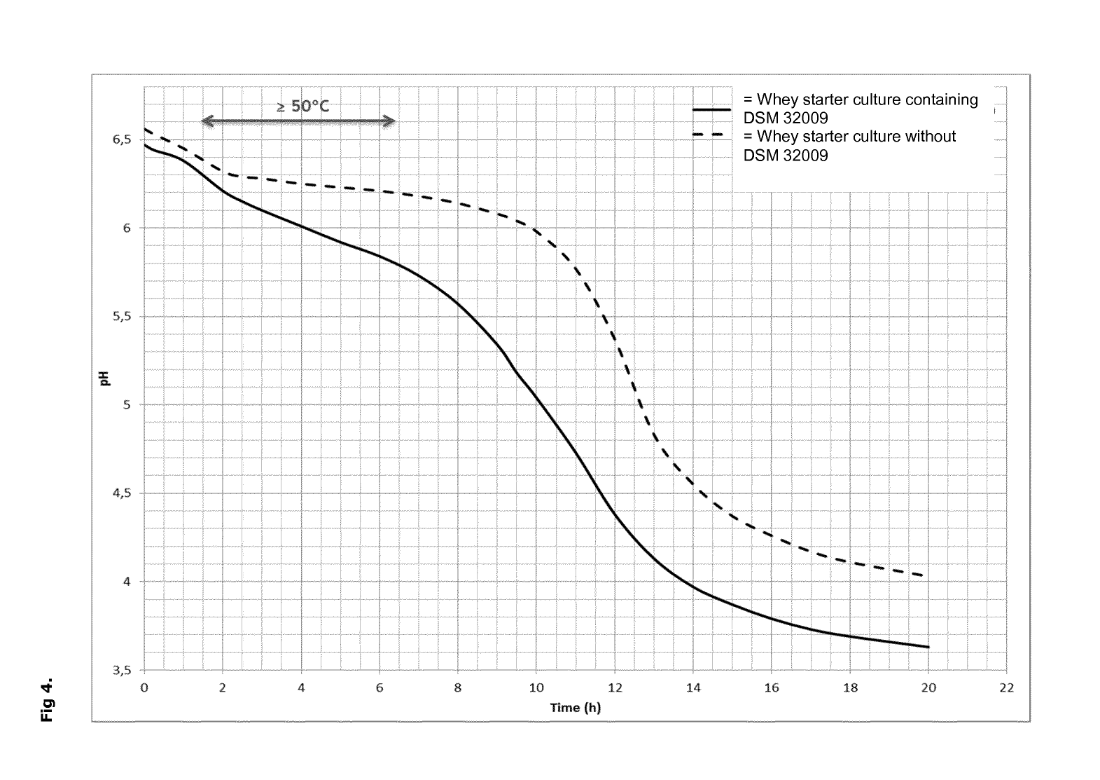

Milk used for acidification experiment was prepared from 9.5% skim milk powder rehydrated in water and subsequently heat treated at 140° C./8 sec and 100° C./30 min. Milk was inoculated with 3% of the whey starter prepared as described in Example 1.a. The pH was measured continuously with pH probes connected to a datalogger as shown in FIG. 4. Temperature compensation was used to secure accurate pH measurement throughout the temperature gradient. Incubation temperature was as described in FIG. 3. During the time where the temperature is above 50° C., the whey starter culture containing the Lactobacillus delbrueckii subsp. lactis DSM 32009 strain decreased the pH of the milk from pH 6.30 to pH 5.80 (deltapH=0.50 pH units). The whey starter culture without the Lactobacillus delbrueckii subsp. lactis DSM 32009 strain decreased the pH of the milk from pH 6.40 to pH 6.20 (deltapH=0.2 pH units). Moreover, it is observed that the whey starter culture containing the Lactobacillus delbrueckii subsp. lactis DSM 32009 strain continues to lower the milk pH after the cooking step while the whey starter culture without the Lactobacillus delbrueckii subsp. lactis DSM 32009 strain has a longer lag phase with no or very little pH drop. As a comparison, the milk acidified with the whey starter culture containing the Lactobacillus delbrueckii subsp. lactis DSM 32009 strain reaches pH 5.0 in 10 hours while the milk acidified with the whey starter culture without the Lactobacillus delbrueckii subsp. lactis DSM 32009 strain reaches pH 5.0 in 12.5 hours.

FIG. 3 shows the temperature profile used for the milk acidification using a whey starter containing the Lactobacillus delbrueckii subsp. lactis DSM 32009 strain. This temperature profile simulates the temperature profile used for the manufacture of Grana Padano cheese. The temperature profile consists of:
A constant temperature of 32° C. for 30 min
A temperature increase to 54° C. and kept constant for 3 hours
A linear temperature decrease to 30° C.

FIG. 4 shows the evolution of the pH in milk inoculated with a whey starter solution containing or not the Lactobacillus delbrueckii subsp. lactis DSM 32009 strain. Temperature gradient is as described in FIG. 3.

### REFERENCES

Rosetti, L., Fornasari, M. E., Gatti, M., Lazzi, C., Neviani, E. and Giraffa, G. Grana Padano cheese whey starters: Microbial composition and strain distribution. International Journal of Food Microbiology 127 (2008) pp. 168-171.

1. A process for producing cheese, comprising:
adding to milk a starter culture comprising an acidifying Lactobacillus strain to obtain a mixture and
heating the mixture to a temperature in the range of 45 to 65° C. or maintaining the mixture at a temperature in the range of 45 to 65° C.

2. A process according to claim 1, wherein the acidifying Lactobacillus is able to acidify the milk by at least 0.4 pH units in 2.5 hours at a temperature around 50° C. when inoculated with a 3% whey starter culture.

3. A process according to claim 2, wherein the acidifying Lactobacillus strain is a Lactobacillus delbrueckii strain or a mutant or variant thereof.

4. A process according to claim 2, wherein the acidifying Lactobacillus strain is a Lactobacillus delbrueckii subsp. lactis strain or a mutant or variant thereof.

5. A process according to claim 2, wherein the acidifying Lactobacillus strain is the Lactobacillus strain deposited as DSM 32009 or a mutant or variant thereof.

6. A process according to claim 1, wherein the acidifying Lactobacillus strain has been modified by an act of man.

7. A process according to claim 1, further comprising adding to the milk one or more additional bacterial strains or cultures.

8. A process according to claim 1, wherein the starter culture comprises one or more bacterial strains selected from Lactococcus lactis, Leuconostoc mesenteroides, Pediococcus pentosaceus, Lactobacillus casei, Lactobacillus paracasei, Streptococcus themophilus, Enterococcus faecium, Lactobacillus delbrueckii subsp. bulgaricus, Lactobacillus delbrueckii subsp. Lactis, Lactobacillus helveticus spp, Lactobacillus delbrueckii, Lactobacillus acidophilus and Propionic bacterium.

9. A process according to claim 8 wherein the starter culture comprises Lactobacillus helveticus spp, Lactobacillus delbrueckii, Streptococcus thermophilus and Lactobacillus casei.

10. A process according to claim 1; further comprising adding to the milk one or more coagulants.

11. A process according to claim 10, wherein the one or more coagulants is selected from bovine derived coagulants or variants thereof, microbial derived coagulants or variants thereof, camel derived coagulants or variants thereof, rennets, and chymosins.

12. A process according to claim 1, wherein the starter culture is a whey starter culture.

13. A process according to claim 12, wherein, before being added to the milk, the whey starter culture is subject to physical conditions comprising a temperature drop from around 48° C. to around 39° C. in 9 hours, a constant temperature of around 39° C. for around 8 hours, and cooling and storing at around 16° C. until it is added to the milk.

14. A process according to claim 1, wherein the starter culture is added as a direct vat set (DVS) culture.

15. A process according to claim 14, wherein the DVS culture is added to a whey starter.

16. A process according to claim 1, wherein the acidifying Lactobacillus strain is added in an amount of at least 5×10¹⁰ CFU/500 liter whey.

17. A process according to claim 14, wherein the DVS culture is added directly to the milk.

18. A process according to claim 1, wherein the starter culture is added to the milk in an amount sufficient to facilitate acidification of the milk by at least 0.4 pH units in 2.5 hours at a temperature around 50° C.

19. A process according to claim 7, wherein the one or more additional bacterial strains or cultures includes a bacterial strain or culture that belongs to the genus Propionibacterium.

20. A cheese obtained by the process of claim 1.

21. A cheese according to claim 20, wherein the cheese is a Grana type cheese.

22. An acidifying Lactobacillus delbrueckii subsp. lactis strain, wherein the strain is the acidifying Lactobacillus delbrueckii subsp. lactis strain deposited as DSM 32009, or a mutant or variant thereof.

23. The acidifying Lactobacillus delbrueckii according to claim 22, wherein the strain is a mutant or variant of the acidifying Lactobacillus delbrueckii subsp. lactis strain deposited as DSM 32009.

24. The acidifying Lactobacillus delbrueckii strain according to claim 22, wherein the strain is able to acidify milk by at least 0.4 pH units in 2.5 hours at a temperature around 50° C. when inoculated with a 3% starter culture.

25. The acidifying Lactobacillus delbrueckii strain according to claim 23, wherein the strain has been modified by an act of man.

26. A composition comprising an acidifying Lactobacillus delbrueckii strain of claim 22.

27. The composition according to claim 26 further comprising one or more excipients.

28. The composition according to claim 27, wherein the one or more excipients is selected from polyhydroxy compounds and their derivatives, trehalose, and dimethyl sulfoxide (DMSO).

29. The composition according to claim 27, wherein the composition is a direct vat set (DVS) starter culture composition.

30. The composition according to claim 29, wherein the DVS starter culture composition comprise at least 1×10̂10 CFU/g.

31. A process for making cheese comprising adding to milk an acidifyng Lactobacillus delbrueckii subsp. lactis strain of claim 22.

32. A process for making cheese, comprising adding to milk a composition of claim 26.
